(19) United States
(12) Patent Application Publication
(10) Pub. No.: US 2021/0247402 A1
(43) Pub. Date: Aug. 12, 2021

(54) IDENTIFICATION OF IMMUNOGLOBULINS USING MASS SPECTROMETRY

(71) Applicant: The Binding Site Group Ltd, Birmingham (GB)

(72) Inventors: Dhananjay SAKRIKAR, Rochester, MN (US); Stephen HARDING, Birmingham (GB); David BARNIDGE, Rochester, MN (US); Michelle LAJKO, Rochester, MN (US)

(21) Appl. No.: 17/052,499

(22) PCT Filed: May 3, 2019
(86) PCT No.: PCT/GB2019/051239

§ 371 Date: Nov. 2, 2020

Related U.S. Application Data
(60) Provisional application No. 62/667,076, filed on May 4, 2018.

(30) Foreign Application Priority Data
May 24, 2018 (GB) 1808529.0

(51) Int. Cl. G01N 33/68; G01N 30/72; G01N 33/574

(52) U.S. Cl. CPC G01N 33/6851; G01N 2800/52; G01N 33/57426; G01N 30/7266

(57) ABSTRACT

This document relates to materials and methods for identifying and/or quantifying immunoglobulins from a biological sample without pre-purification of the immunoglobulins prior to ionization and detection using mass spectrometry. For example, a monoclonal light chain from a monoclonal immunoglobulin may be observed using matrix assisted laser desorption ionization-time of flight (MALDI-TOF) mass spectrometry after diluting a sample containing the monoclonal immunoglobulin with an aqueous buffer containing acid and a reducing agent then mixing the sample with alpha-cyano-4-hydroxycinnamic acid matrix (CHCA). In another example, an intact monoclonal immunoglobulin may be observed in a sample using MALDI-TOF mass spectrometry after diluting the sample containing the monoclonal immunoglobulin with water then mixing the sample with CHCA matrix.

### BACKGROUND

### 1. Technical Field

This document relates to materials and methods for identifying and/or quantifying immunoglobulins from a biological sample without pre-purification of the immunoglobulins prior to ionization and detection using mass spectrometry. For example, a monoclonal light chain from a monoclonal immunoglobulin may be observed using matrix assisted laser desorption ionization-time of flight (MALDI-TOF) mass spectrometry after diluting a sample containing the monoclonal immunoglobulin with an aqueous buffer containing acid and a reducing agent then mixing the sample with alpha-cyano-4-hydroxycinnamic acid matrix (CHCA). In another example, an intact monoclonal immunoglobulin may be observed in a sample using MALDI-TOF mass spectrometry after diluting the sample containing the monoclonal immunoglobulin with water then mixing the sample with CHCA matrix.

### 2. Background Information

Human immunoglobulins contain two identical heavy chain polypeptides and two identical light chain polypeptides bound together by disulfide bonds. There are two different light chain isotypes (kappa and lambda) and five different heavy chain isotypes (IgG, IgA, IgM, IgD, and IgE). A monoclonal immunoglobulin, polyclonal immunoglobulins, and any combination of the light chain and/or heavy chain from the monoclonal or polyclonal immunoglobulins, can be identified using a mass spectrometer by way of accurate molecular mass.

The detection and monitoring of immunoglobulins by mass spectrometry is generally known in the art.

For example, WO 2015/154052A and WO2014/150170 disclose enriching immunoglobulins from a sample of serum using Melon Gel, prior to liquid chromatography and ESI-Q-TOF quadrupole time-of-flight mass spectrometry. WO 2015/154052A similarly uses Melon Gel purification with LC-ESI-Q-TOF mass spectrometry. Barnidge D. R. et al (J. Neuroimmunology (2015), 285, 123-126) also describes enriching immunoglobulins from serum samples. Samples of purified serum were then reduced using DTT (dithiothreitol) prior to analysis by LC-MS.

Mills et al (Clin. Chem. (2016) 62(10) 1334-1344) describes using camelid-derived nanobodies against the constant regions of heavy chains or the light chain constant domains to purify antibodies, prior to MALDI-TOF.

These examples show that conventionally antibodies are purified or enriched prior to analysis by mass spectrometry.

The complex purification or enrichments of immunoglobulins prior to mass spectrometry (M.S.) increases the time to study immunoglobulins in samples.

Hortin G. L. and Remaley A. T. (Clin. Geonomics (2006) 103-114) describe the determination of the mass of major plasma proteins and serum samples are described. Specimens were diluted with 10 mmol/L ammonium acetate and 10 g/L sinapinic acid in 40% acrylonitrile/10% ethanol/50% water/0.1% trifluoroacetic acid.

A wide range of purified proteins were anaylsed, including glycoproteins, transferrin, immunoglobulin G, apolipoproteins and transthyretin.

Diluted serum samples for two specimens were able to identify IgG, albumin, pre albumin, transferrin and apolipoprotein. The paper concluded that for several of the most abundant proteins of masses less than 30,000 (i.e. excluding for example IgG with a mass of 147,000), mass measurements could be made with sufficient precision and accuracy to allow detection of chemical modifications and sequence modifications. The paper also notes that there were drawbacks in the analysis of complex mixtures without fractionation. Firstly, only the most abundant components could be observed. Secondly, there was a general suppression of signals by the high protein and salt concentrations. Albumin was observed to interfere with the ability to detect other peaks with m/z 30,000. The paper concluded that selective depletion of IgG and albumin should be used. No identification of other immunoglobulins or indeed light chains was disclosed.

There are a number of proliferative diseases associated with antibody producing cells.

In many such proliferative diseases a plasma cell proliferates to form a monoclonal tumour of identical plasma cells. This results in production of large amounts of identical immunoglobulins and is known as a monoclonal gammopathy.

Diseases such as myeloma and primary systemic amyloidosis (AL amyloidosis) account for approximately 1.5% and 0.3% respectively of cancer deaths in the United Kingdom. Multiple myeloma is the second-most common form of haematological malignancy after non-Hodgkin lymphoma. In Caucasian populations the incidence is approximately 40 per million per year. Conventionally, the diagnosis of multiple myeloma is based on the presence of excess monoclonal plasma cells in the bone marrow, monoclonal immunoglobulins in the serum or urine and related organ or tissue impairment such as hypercalcaemia, renal insufficiency, anaemia or bone lesions. Normal plasma cell content of the bone marrow is about 1%, while in multiple myeloma the content is typically greater than 10%, frequently greater than 30%, but may be over 90%.

AL amyloidosis is a protein conformation disorder characterised by the accumulation of monoclonal free light chain fragments as amyloid deposits. Typically, these patients present with heart or renal failure but peripheral nerves and other organs may also be involved.

There are a number of other diseases which can be identified by the presence of monoclonal immunoglobulins within the blood stream, or indeed urine, of a patient. These include plasmacytoma and extramedullary plasmacytoma, a plasma cell tumour that arises outside the bone marrow and can occur in any organ. When present, the monoclonal protein is typically IgA. Multiple solitary plasmacytomas may occur with or without evidence of multiple myeloma. Waldenström's macroglobulinaemia is a low-grade lymphoproliferative disorder that is associated with the production of monoclonal IgM. There are approximately 1,500 new cases per year in the USA and 300 in the UK. Serum IgM quantification is important for both diagnosis and monitoring. B-cell non-Hodgkin lymphomas cause approximately 2.6% of all cancer deaths in the UK and monoclonal immunoglobulins have been identified in the serum of about 10-15% of patients using standard electrophoresis methods. Initial reports indicate that monoclonal free light chains can be detected in the urine of 60-70% of patients. In B-cell chronic lymphocytic leukaemia monoclonal proteins have been identified by free light chain immunoassay.

Additionally, there are so-called MGUS conditions. These are monoclonal gammopathy of undetermined significance. This term denotes the unexpected presence of a monoclonal intact immunoglobulin in individuals who have no evidence of multiple myeloma, AL amyloidosis, Waldenström's macroglobulinaemia, etc. MGUS may be found in 1% of the population over 50 years, 3% over 70 years and up to 10% over 80 years of age. Most of these are IgG- or IgM-related, although more rarely IgA-related or bi-clonal. Although most people with MGUS die from unrelated diseases, MGUS may transform into malignant monoclonal gammopathies.

In at least some cases for the diseases highlighted above, the diseases present abnormal concentrations of monoclonal immunoglobulins or free light chains. Where a disease produces the abnormal replication of a plasma cell, this often results in the production of more immunoglobulins by that type of cell as that “monoclone” multiplies and appears in the blood.

### SUMMARY

This document relates to materials and methods for identifying and/or quantifying immunoglobulins from a biological sample without pre-purification of the immunoglobulins prior to ionization and detection using mass spectrometry. In some cases, mass spectrometry techniques can be used to identify and/or quantify immunoglobulins in a biological sample without the need for additional purification of the immunoglobulins either by immunopurification or removal of other non-immunoglobulin proteins from the sample. As demonstrated herein, a monoclonal immunoglobulin, or polyclonal immunoglobulins, and any combination of the light chain and/or heavy chain from the monoclonal or polyclonal immunoglobulins, can be identified by dilution of the sample in a buffer with or without a reducing agent. This methodology is faster to perform than other methods that employ purification prior to ionization and detection using mass spectrometry reducing costs and increasing throughput.

The Applicant has unexpectedly found that it is possible to detect and quantify immunoglobulins even in relatively complex samples, such as blood, serum, plasma or cerebrospinal fluid by a dilution of the sample or even reconstitution of a dried sample, using water or an aqueous buffer

The invention provides a method for identifying and/or quantifying monoclonal and/or polyclonal immunoglobulins in the sample comprising the steps of:
(i) providing a sample containing an immunoglobulin from a subject;
(ii) diluting the sample with water or an aqueous buffer to form a diluted sample;
(iii) ionizing the diluted sample and detecting and optionally quantifying intact immunoglobulin, or a light chain or a heavy chain of the immunoglobulin, by mass spectrometry.

Mass spectrometry may potentially be any mass spectrometry technique. This includes, for example, quadropole time-of-flight mass spectrometry, for example in combination with liquid chromatography, such as liquid chromatography-electrospray ionization-mass spectrometry (LC-ESI-MS). However, more typically the mass spectrometry is matrix assisted laser desorption time-of-flight (MALDI-TOF) mass spectrometry. As explained above, the unpurified diluted sample is introduced into the mass spectrometry system, meaning that all components of the sample are introduced into the system at the same time. In embodiments of the invention where liquid chromatography-mass spectrometry (LC-MS) is used the liquid chromatography column simply separates the components of the sample so that they arrive at the mass spectrometer at different times, depending on how they interact with the liquid chromatography column.

Where the mass spectrometry based technique includes the use of a matrix, the diluted sample may be mixed with a suitable matrix prior to being ionized. For example, the matrix may be alpha-cyano-4-hydroxycinnamic acid (CHCA) matrix mixed with acetonitrile and water containing an acid such as trifluoroacetic acid. Other matrices include, for example, a mixture of sinapinic acid, or 2,5-Dihydroxybenzoic acid.

The sample buffer may be any suitable aqueous buffer, but includes, for example, buffers containing a mixture of an acid, such as acetic acid, and a reducing agent, such as TCEP (tris(2-carboxyethyl)phosphine), TCEP-HCl, 2-Mercaptoethanol (BME) or dithiothreitol (DTT). In preferred embodiments of the invention the buffer may contain acetic acid and TCEP-HCl, for example, the buffer may contain 5% acetic acid (v/v) and 20 mM TCEP-HCl. Alternatively, the sample may be reduced using a reducing agent, such as DTT, and then mixed with an acidic aqueous solution. For example, the sample may be reduced with 10 mM DTT and then mixed with the acidic aqueous solution.

Heavy chains that are attached to light chains may be detached from one another by including a reducing agent in the buffer or as a separate addition to the sample. The reducing agent separates the heavy chains from the light chains and allows the separate heavy chains and light chains to be detected and/or quantified. Suitable reducing agents include those generally known in the art, such as DTT, for example, at 200 mM. The heavy chains detected may be IgG, IgA, IgM, IgD or IgM. The light chains may be kappa or lambda light chains.

Typically the intact immunoglobulin, the intact light chains, or the intact heavy chains are not fragmented using specific reagents prior to mass spectrometry. For example, the immunoglobulins are not typically enzymatically digested with a specific protease prior to mass spectrometry.

The immunoglobulin is not typically enriched or purified, for example, by affinity purification prior to mass spectrometry. For example, the immunoglobulin is not typically immunopurified by using anti-heavy class and/or light chain type antibodies, such as anti-IgG, anti-IgA, anti-IgM anti-kappa or anti-lambda antibodies. Typically the immunoglobulin is not purified with, for example, Melon Gel, Protein A or Protein G. Typically the immunoglobulins are not purified by, for example, chromatography such as size exclusion chromatography.

Where a sample of blood or tissue is provided, cells such as red blood cells and/or white blood cells may be removed, for example by centrifugation, prior to dilution.

The sample may be selected from, for example, serum, plasma, blood, urine and cerebrospinal fluid, especially blood, plasma or serum. The sample may be from a human subject.

The sample may be from a subject, such as a human subject, who has, or is suspected of having, a proliferative disease associated with plasma producing cells. Those proliferative diseases include those described above, such as monoclonal gammopathies. These include, for example, myeloma and AL amyloidosis, and other such diseases as described above.

The sample may be a dried or at least partially dried sample that is rehydrated with the water or aqueous buffer. This may have implications in allowing the storage of the sample in a dried state or allow recovery of a dried sample from the subject on an article, such as clothing. Further processing of the sample is typically not needed.

Typically the immunoglobulin detected and/or quantified is a monoclonal immunoglobulin, monoclonal heavy chain or monoclonal light chain. As shown in the attached figures, monoclonal immunoglobulins produce a distinct peak above the background polyclonal antibody production. This may be readily detected and/or quantified by the method of the invention. Alternatively, polyclonal heavy chains, polyclonal light chains, and polyclonal intact immunoglobulins may be detected and quantified.

Alternatively, the relative amounts of kappa and lambda light chains may be quantified to determine the ratio of kappa to lambda light chains.

Typically the light chains are free light chains.

A sample is usually, but not always, diluted prior to analysis by mass spectrometry. A typical dilution is 1:1,280 but may range between 1:50 and 1:5000, more typically between 1:500 and 1:3000 or 1:1000 and 1:2000 prior to detection by mass spectrometry.

The invention provides a rapid way of detecting the presence or absence of, for example, monoclonal immunoglobulins using mass spectrometry, without the need for complex additional purification techniques of the sample.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows 8 mass spectra obtained by; 1) serially diluting a monoclonal IgA1 Kappa standard (concentration 31 g/L) in aqueous buffer containing 5% acetic acid and 20 mM tris(2-carboxyethyl)phosphine hydrochloride (TCEP); 2) mixing the diluted sample with CHCA matrix; 3) analyzing the sample using MALDI-TOF mass spectrometry. The mass spectra cover an m/z range of 10,500 to 13,000 which includes the +2 charge state of the monoclonal kappa light chain. The light chain is labeled in the 1 to 1,280 dilution in FIG. 1 and is also clearly observed in the 1 to 160, 1 to 320, 1 to 640, and 1 to 2,560 dilutions.

FIG. 2 shows a plot of the intensity of the +2 charge state from the serial dilution of IgA1 Kappa standard in aqueous buffer containing 5% acetic acid and 20 mM TCEP. The plot demonstrates that the intensity of the signal from the monoclonal kappa light chain increases as the sample is diluted up to 1 to 1,280 and then decreases in subsequent dilutions. The observation that the signal from the +2 charge state from the monoclonal kappa light chain increases as the sample is diluted is related to the ratio of matrix to total protein in the sample.

FIG. 3 shows the MALDI-TOF mass spectrum of the 1 to 1,280 dilution from FIG. 1 over the m/z range 7,000 to 25,000. The +1, +2, and +3 charge states from the monoclonal kappa light chain are labeled in the figure along with the +3, +4, +5, and +6 charge states from serum albumin. The figure demonstrates the ability to observe a monoclonal light chain in the presence of serum albumin the most abundant protein in serum.

FIG. 4 shows the MALDI-TOF mass spectrum of a sample from a patient with an IgG kappa M-protein (concentration 4.0 g/L) diluted 1 to 1,280 in aqueous buffer containing 5% acetic acid and 20 mM TCEP. The +2 charge state ion from the monoclonal kappa light chain is observed at 11,724.196 m/z. The +4 charge state ion from serum albumin is also labeled in the figure.

FIG. 5 shows the MALDI-TOF mass spectrum of a sample from a patient with an IgG lambda M-protein (concentration 6.0 g/L) diluted 1 to 1,280 in aqueous buffer containing 5% acetic acid and 20 mM TCEP. The +2 charge state ion from the monoclonal lambda light chain is observed at 11,467.0 m/z. The +4 charge state ion from serum albumin and the polyclonal kappa molecular mass distribution are also labeled in the figure.

FIG. 6 shows the MALDI-TOF mass spectrum of a sample from a patient with an IgA kappa M-protein (concentration 37 g/L) diluted 1 to 1,280 in aqueous buffer containing 5% acetic acid and 20 mM TCEP. The +2 charge state ion from the monoclonal kappa light chain is observed at 11,891.836 m/z.

FIG. 7 shows the MALDI-TOF mass spectrum of a sample from a patient with an IgA lambda M-protein that was not quantified by serum protein electrophoresis diluted 1 to 1,280 in aqueous buffer containing 5% acetic acid and 20 mM TCEP. The +2 charge state ion from the monoclonal lambda light chain labeled at 11,139.298 m/z is labeled in the figure.

FIG. 8 shows the MALDI-TOF mass spectrum of a sample from a patient with an IgM kappa M-protein (concentration 8.0 g/L) diluted 1 to 1,280 in aqueous buffer containing 5% acetic acid and 20 mM TCEP. The +2 charge state ion from the monoclonal kappa light chain is observed at 11,746.744 m/z.

FIG. 9 shows the MALDI-TOF mass spectrum of a sample from a patient with an IgM lambda M-protein (concentration 7.0 g/L) diluted 1 to 1,280 in aqueous buffer containing 5% acetic acid and 20 mM TCEP. The +2 charge state ion from the monoclonal lambda light chain is observed at 11,350.780 m/z.

FIG. 10 shows MALDI-TOF mass spectra from a patient with an IgG kappa M-protein (concentration 20 g/L) (top mass spectrum) and normal human serum (bottom mass spectrum) each diluted 1 to 200 in water. Since each sample was diluted in water, without acid or a reducing agent, the +3 charge state of the intact monoclonal IgG kappa, with the heavy and light chains still connected by disulphide bonds, is observed at 51,150.933 m/z (top mass spectrum). No peak is present in the mass spectrum from the normal human serum (bottom mass spectrum) at this molecular mass.

FIG. 11 shows MALDI-TOF mass spectra from a patient with an IgG kappa M-protein (concentration 20 g/L) (top mass spectrum) and normal human serum (bottom mass spectrum) each diluted 1 to 200 in water. Since each sample was diluted in water, without acid or a reducing agent, the +3 charge state of the intact monoclonal IgG kappa, with the heavy and light chains still connected by disulphide bonds, is observed at 49,027.884 m/z (top mass spectrum). No peak is present in the mass spectrum from the normal human serum (bottom mass spectrum) at this molecular mass.

FIG. 12 shows reconstituted dried samples of healthy blood and blood spiked with 10 g/L IgGkappa myeloma, including singly charged (+1) peaks (FIG. 12A) and doubly charged peaks (FIG. 12B).

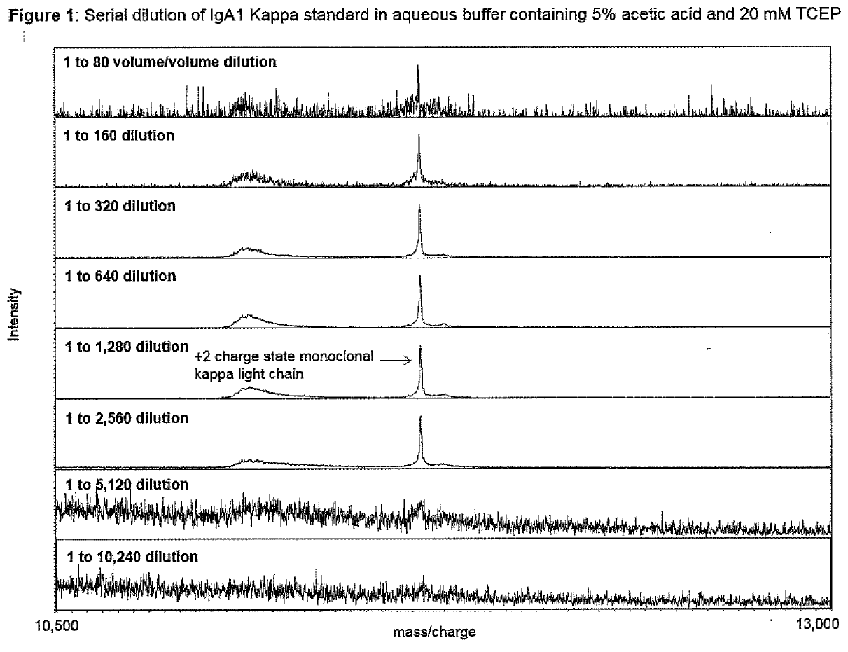

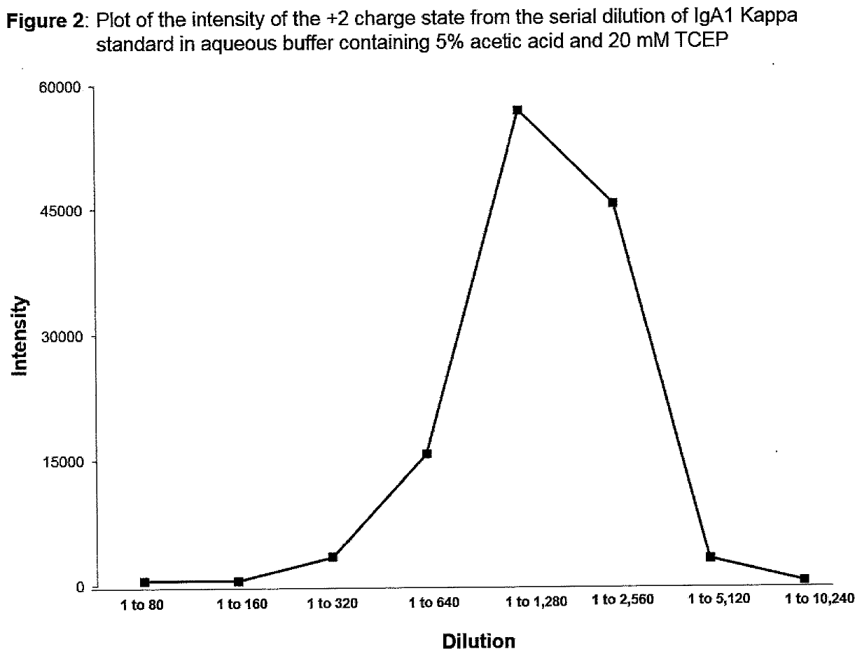

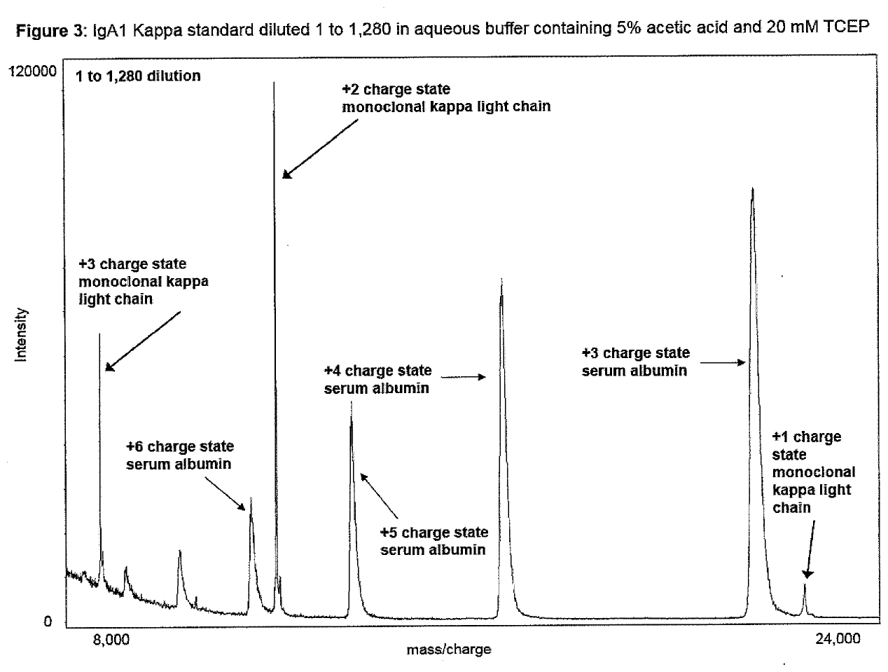

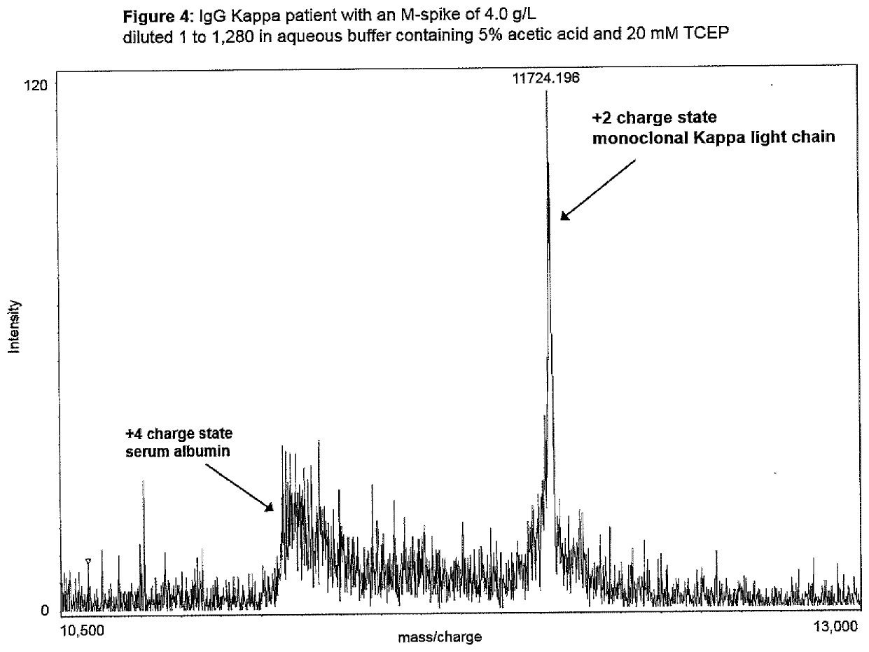

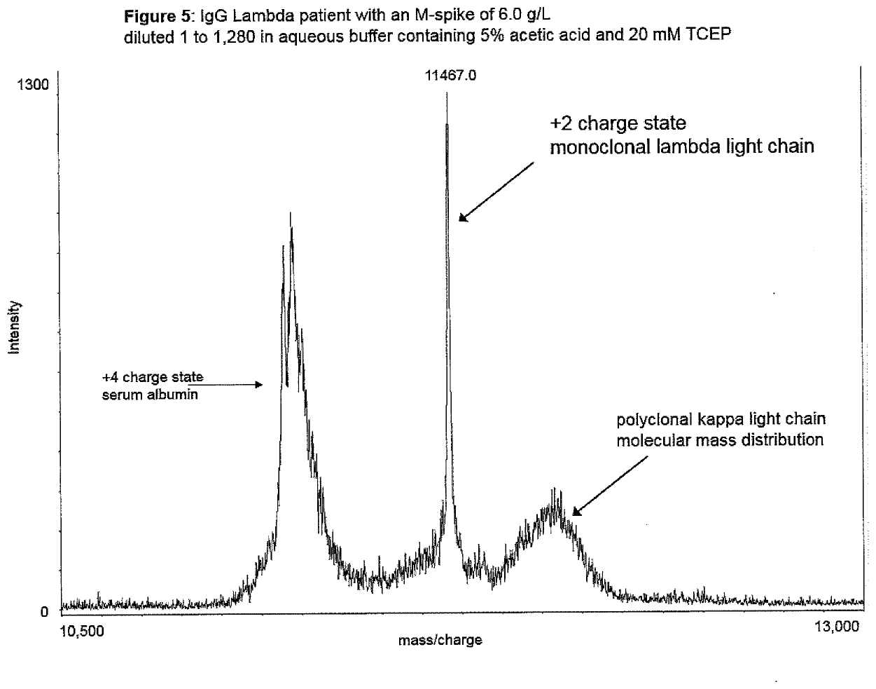

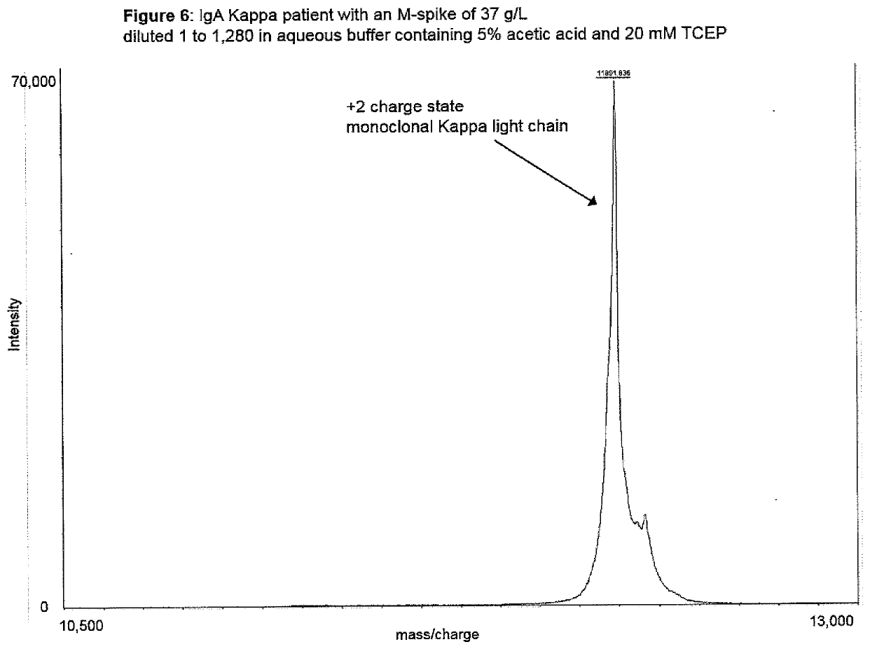

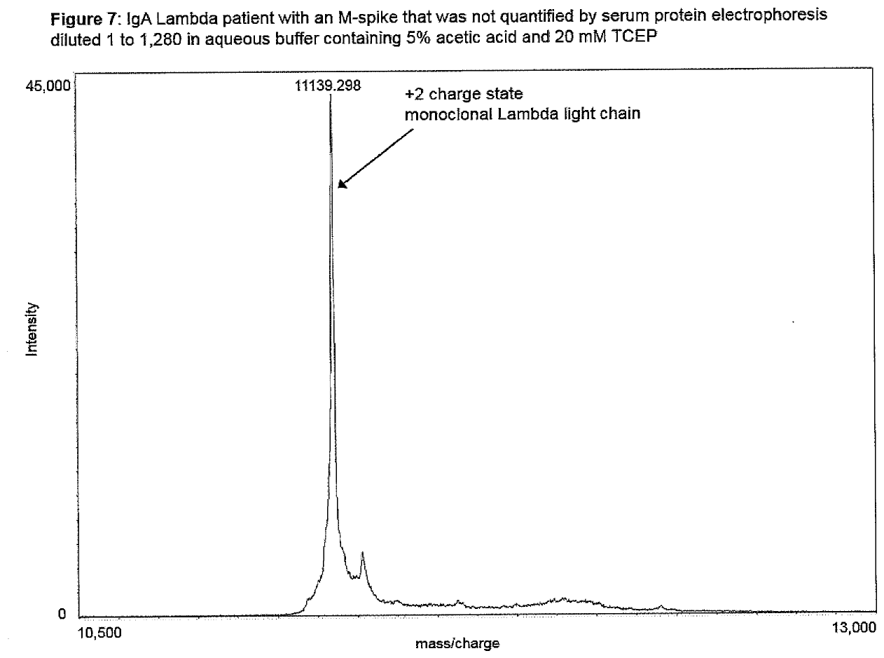

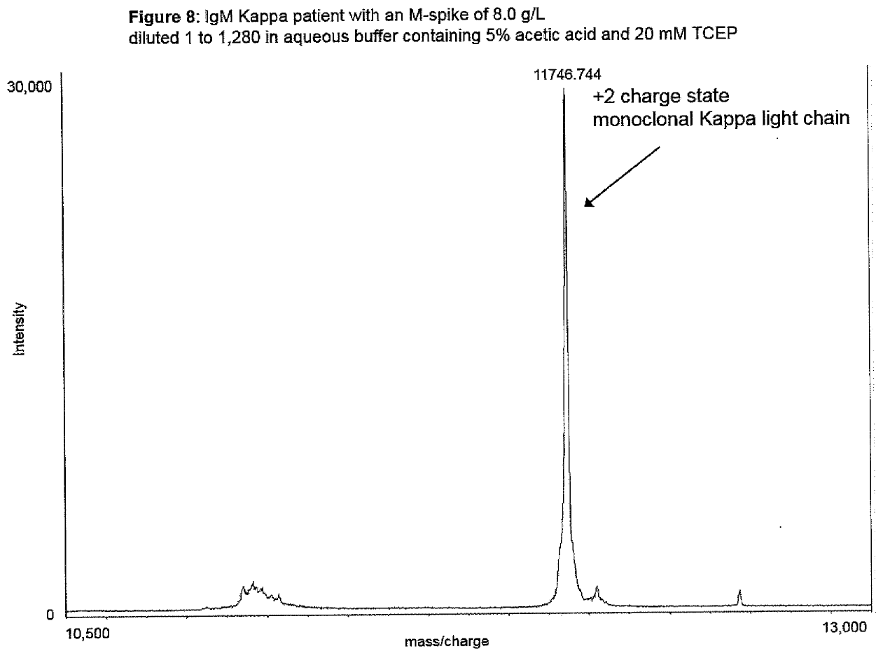

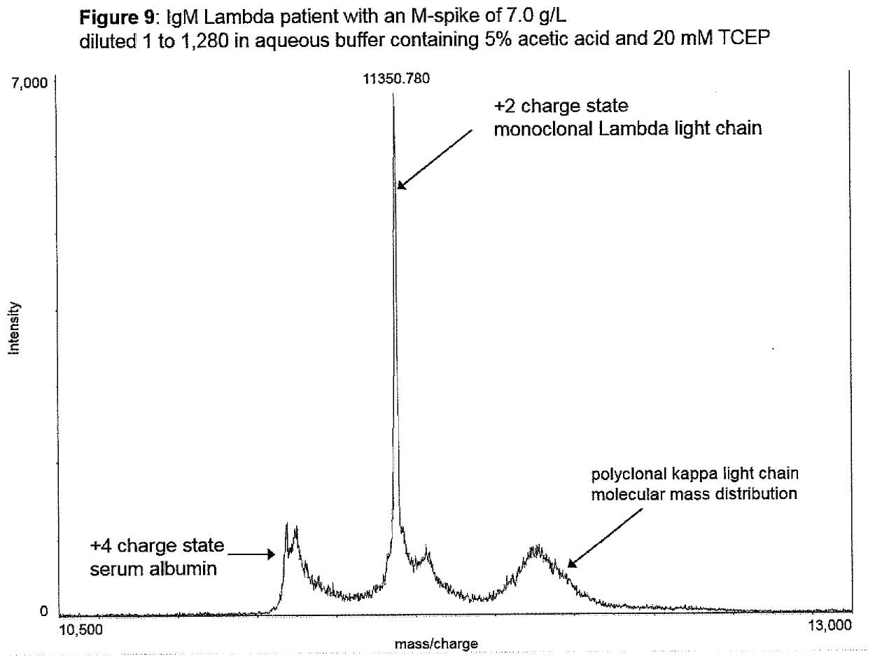

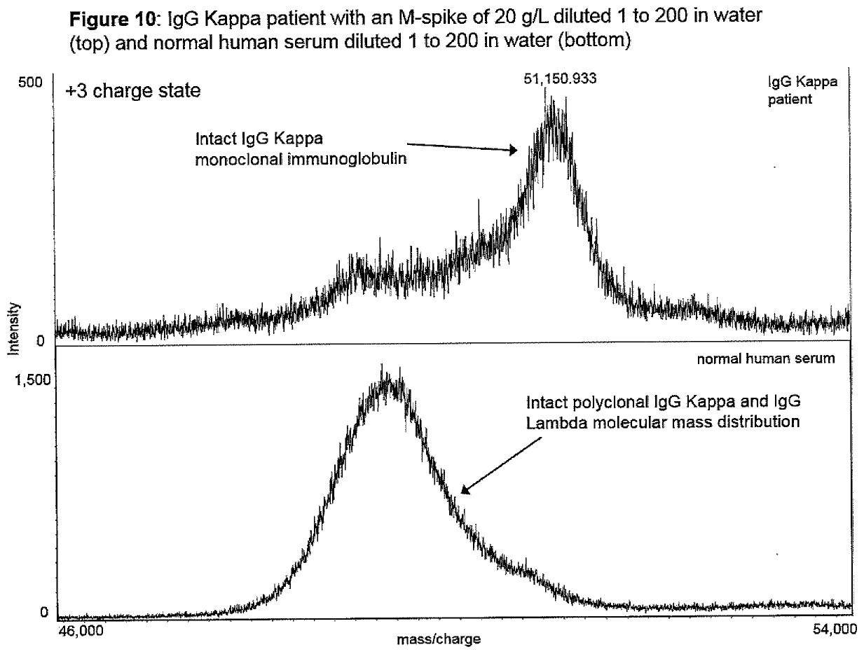

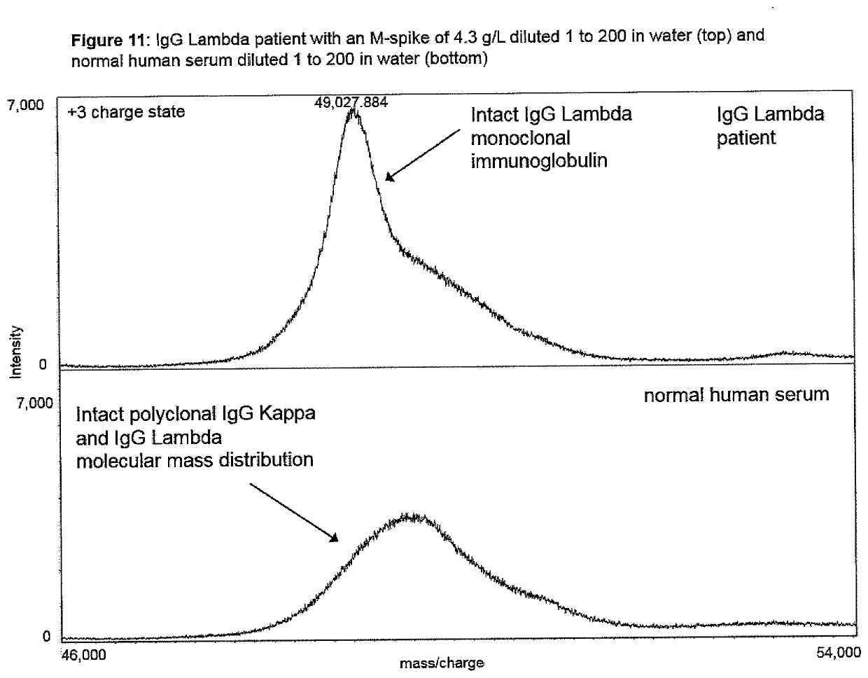

### SAMPLES AND SAMPLE PREPARATION

The materials and methods for identifying and quantifying a monoclonal immunoglobulin or polyclonal immunoglobulins as described herein can include any appropriate sample. A sample can be any biological sample, such as a tissue (e.g., adipose, liver, kidney, heart, muscle, bone, or skin tissue) or biological fluid (e.g., blood, serum, plasma, urine, lachrymal fluid, or saliva). The sample can be from a patient that has immunoglobulins, which includes but is not limited to a mammal, e.g. a human, dog, cat, primate, rodent, pig, sheep, cow, horse, bird, reptile, or fish. A sample can also be a man-made reagent, such as a mixture of known composition or a control sample. In some cases, the sample is serum from a human patient.

### Mass Spectrometry Methods

The materials and methods for identifying and quantifying a monoclonal immunoglobulin or polyclonal immunoglobulins as described herein can include any appropriate mass spectrometry (MS) technique. In some cases, matrix assisted laser adsorption ionization (MALDI) Time-of-Flight (TOF) MS can be used to analyze the mass spectrum of a sample.

The invention will be further described in the following examples, which do not limit the scope of the invention described in the claims.

### EXAMPLES

### Example 1

### Dilution of a Serum Sample in an Aqueous Buffer Containing Acid and a Reducing Agent then Analyzing the Sample Using MALDI-TOF MS

Monoclonal and/or polyclonal light chains can be identified and/or quantified in a sample using matrix assisted laser desorption ionization-time of flight (MALDI-TOF) mass spectrometry by first diluting the sample containing the immunoglobulin(s) with an aqueous buffer containing acid and a reducing agent then mixing the sample with a MALDI matrix such as alpha-cyano-4-hydroxycinnamic acid matrix (CHCA).

### Example 2

### Dilution of a Serum Sample in Water then Analyzing the Sample Using MALDI-TOF MS

An intact monoclonal immunoglobulin and/or intact polyclonal immunoglobulins can be identified and/or quantified observed in a sample using matrix assisted laser desorption ionization-time of flight (MALDI-TOF) mass spectrometry by first diluting the sample containing the immunoglobulin(s) with water then mixing the sample with a MALDI matrix such as alpha-cyano-4-hydroxycinnamic acid matrix (CHCA).

### Example 3

### Whole Blood Dried Spot Analysis

### Methods.

Freshly drawn capillary blood from a finger prick, was mixed 1+1 with deionised water with or without the presence of a myeloma IgG kappa paraprotein. 10 μl was spotted onto 3 MM filter paper (Whatman) and air-dried for 15 min. The filter paper containing the whole blood spots was stored at −80° C. for up to 3 days.

Prior to extraction the dried blood spots were removed from the freezer and allowed the warm up to ambient temperature. The dried blood spots were cut from the filter paper, carefully placed in a 1.5 ml eppendorf tube and then extracted for 30 min with 100 μl reduction-elution buffer (5% acetic acid containing 20 mM TCEP). The extracted liquid material was removed following a centrifugal pulse. Spotting of the extract was performed using an automated liquid handling system (Mosquito) on to a MALDI steel target plate using a semi-wet sandwich method. 1 μl of α-cyano-4-hydroxycinnamic acid, matrix solution (CHCA, 10 mg/ml) was applied first and allowed to dry. Next 1 μl of the extracted sample followed by 1 ul of (CHCA) was applied together and allowed to dry for 15 min. Mass spectra were acquired on a matrix assisted laser desorption ionisation time-of-flight mass spectrometry (MALDI-TOF-MS) system in positive ion mode covering the m/z range of 5000 to 32,000 Da.

Results.

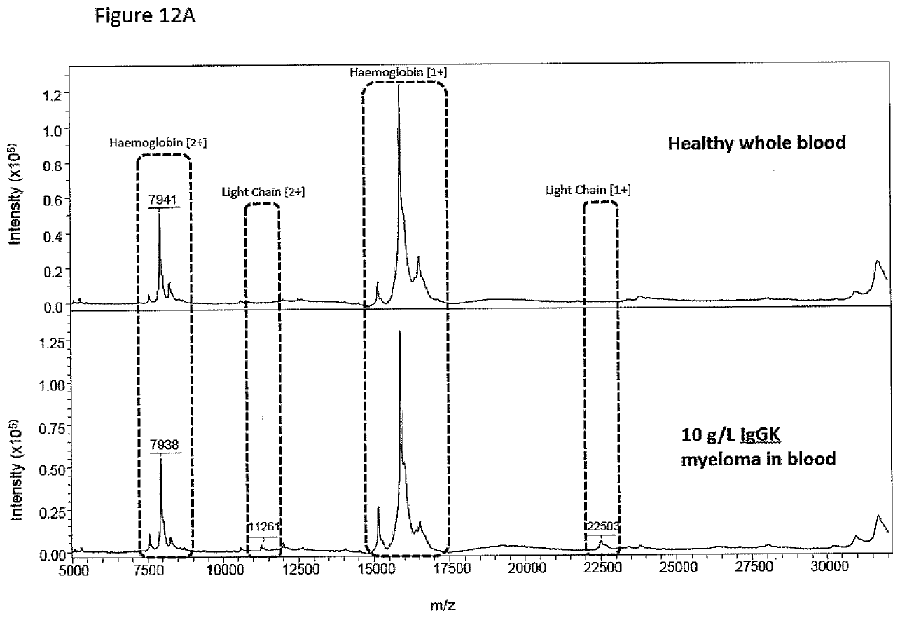

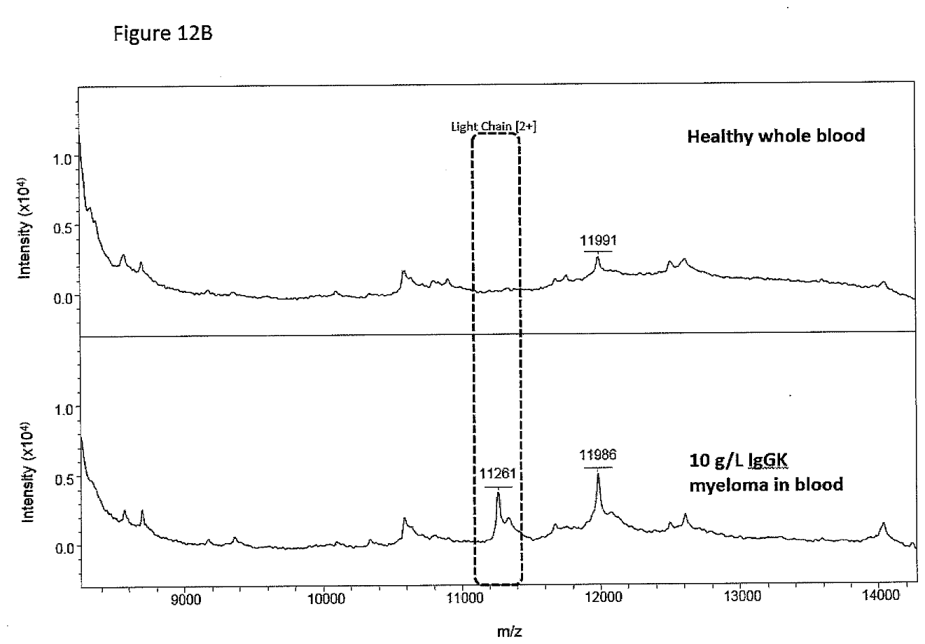

Healthy whole blood spectra showed strong signals for haemoglobin which includes the singly charged (+1, 15 870 m/z) and doubly charged (+2, m/z 7940) ions originating from the beta chain (FIG. 12A). When the blood was supplemented with a IgGK paraprotein at 10 g/L additional signals originating from the kappa light chain were observed including the singly charged (+1, m/z 22508) and doubly charged (+2, m/z 11261) ions (FIGS. 12A and B).

### CONCLUSIONS

Whole dried blood spots can be obtained, eluted and analysed using MALDI-TOF MS without the requirement for sample processing or enrichment

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for identifying and/or quantifying monoclonal and/or polyclonal immunoglobulins in a sample comprising the steps of:
(i) providing a sample of blood, serum, plasma or cerebrospinal fluid containing an immunoglobulin from a subject;
(ii) diluting the sample with water or an aqueous buffer to form a diluted sample;
(iii) ionizing the diluted ample and detecting and optionally quantifying intact immunoglobulin or a light chain or a heavy chain of the immunoglobulin by mass spectrometry.

2. A method according to claim 1 wherein the sample is at least partially dried and is rehydrated with the water or aqueous buffer.

3. A method according to claim 1, wherein the immunoglobulin is not enriched prior to detecting and optionally quantifying the intact immunoglobulin or light chain or heavy chain by mass spectrometry.

4. A method according to claim 3 wherein the immunoglobulins are not enriched by affinity purification or size exclusion chromatography.

5. A method according to claim 1, wherein the mass spectrometry is liquid chromatography-electrospray ionization-mass spectrometry (LC-ESI-MS).

6. A method according to claim 1, wherein the diluted sample is mixed with a matrix, prior to being ionized.

7. A method according to claim 1, comprising treating the sample with a reducing agent, to separate light chains and heavy chains of immunoglobulins prior to detection and/or quantifying of the separated heave chain or light chain by mass spectrometry.

8. A method according to claim 1, wherein the light chain is kappa or lambda light chain.

9. A method according to claim 1, wherein the mass spectrometry is matrix assisted laser desorption time-of-flight (MALDI-TOF) mass spectrometry.

10. A method according to claim 1, wherein the intact immunoglobulin, light chain or heavy chain is not fragmented prior to mass spectrometry.

11. A method according to claim 6, wherein the matrix is a MALDI matrix.

12. A method according to claim 1, wherein the sample is from a subject who has, or who is suspected as having a proliferative disease associated with plasma producing cells.

13. A method according to claim 12, wherein the immunoglobulin detected and/or quantified is a monoclonal immunoglobulin, monoclonal heavy chain or monoclonal light chain.

14. A method according to claim 1, where the sample is diluted by between 1:50 and 1:5000 prior to detection by mass spectrometry.

15. A method of diagnosing or prognosing a proliferative disease associated with plasma producing cells, comprising the use of a method according to claim 1.
